(19) United States
(12) Patent Application Publication
(10) Pub. No.: US 2024/0299715 A1
(43) Pub. Date: Sep. 12, 2024

(54) MAGNETICALLY DRIVEN CAPSULE ROBOT WITH CONTROLLABLE DRUG ADMINISTRATION AND SAMPLING FUNCTIONS AND FABRICATION METHOD THEREOF

(71) Applicant: HUAZHONG UNIVERSITY OF SCIENCE AND TECHNOLOGY, Hubei (CN)

(72) Inventors: Quanliang Cao, Wuhan (CN); Yuxuan Sun, Wuhan (CN); Liang Li, Wuhan (CN); Wang Zhang, Wuhan (CN); Enmin Song, Wuhan (CN)

(73) Assignee: HUAZHONG UNIVERSITY OF SCIENCE AND TECHNOLOGY, Hubei (CN)

(21) Appl. No.: 18/593,968

(22) Filed: Mar. 3, 2024

(30) Foreign Application Priority Data
Mar. 6, 2023 (CN) 202310232473.3
Mar. 6, 2023 (CN) 202310232480.3

(51) Int. Cl. A61M 31/00; A61M 39/22

(52) U.S. Cl. CPC A61M 31/002; A61M 39/22; A61M 2039/226; A61M 2205/0272; A61M 2205/50; A61M 2207/00

(57) ABSTRACT

The disclosure belongs to the technical field of capsule robotics, and in particular, relates to a magnetically driven capsule robot with controllable drug administration and sampling functions and a fabrication method thereof. A magnetically driven capsule robot provided by the disclosure includes a robot body and a magnetic field-driven control module. The robot body includes a capsule shell, two ends of the capsule shell are provided with hollow air chambers, and a carrier chamber is disposed between the hollow air chambers disposed on the two ends. A substance transfer channel, a magnetic lock disposed around the substance transfer channel, and a magnetic soft switch valve matched with the magnetic lock are disposed on a side wall of the carrier chamber. The soft valve automatically closes due to the magnetic attraction force between the lock and valve, and can be flexibly opened under a magnetic torque acting on the valve.

### CROSS-REFERENCE TO RELATED APPLICATION

This application claims the priority benefit of China application serial no. 202310232473.3, filed on Mar. 6, 2023 and China application serial no. 202310232480.3, filed on Mar. 6, 2023. The entirety of each of the above-mentioned patent applications is hereby incorporated by reference herein and made a part of this specification.

### BACKGROUND

### Technical Field

The disclosure belongs to the technical field of magnetic robotics and capsule robotics, and in particular, relates to a magnetically driven capsule robot with controllable drug administration and sampling functions and a fabrication method thereof.

### Description of Related Art

The gastrointestinal (GI) tract, a major hormone-producing organ in the body, is implicated in various clinical disorders including inflammation, ulcers, hemorrhage, and cancers. However, the most commonly used flexible endoscopy faces significant limitations necessitating urgent innovative solutions: while it allows for both diagnosis and treatment integration, it often causes intense discomfort and has contraindications.

Integrating traditional capsules with energy-driven micro-robotics, particularly using magnetic actuation, offers promising solutions for diagnostic and therapeutic challenges. This method has significant advantages such as non-contact, high controllability, and good penetration performance, making it one of the safest and most efficient options for capsules.

An encouraging advancement in this area for GI disease diagnosis is magnetic capsule endoscopy, which has bolstered confidence in the development and biomedical applications of magnetically driven capsules. However, current magnetic capsule endoscopy is limited as it primarily focuses on image acquisition and transmission without additional diagnostic or treatment capabilities.

While certain progress has been made in developing new magnetically driven capsules for drug delivery or sampling purposes, the inherent constraints of the capsule's narrow spatial structure and the complex environment of the GI tract give rise to several ongoing challenges. These include issues such as limited active mobility, complex structure and control, and constrained medical functionalities.

### SUMMARY

In view of the defects found in the related art, the disclosure aims to provide a magnetically driven capsule robot with both controllable drug administration and sampling functions and a fabrication method thereof, so as to solve the problems of limited medical functions, low administration efficiency, complex capsule structure, and complex control found in a magnetically driven capsule robot provided by the related art.

To achieve the above, the disclosure provides a magnetically driven capsule robot including a robot body and a magnetic field-driven control module.

The robot body includes a capsule shell, and two ends of the capsule shell are provided with hollow air chambers. A carrier chamber is disposed between the hollow air chambers, and the carrier chamber and the hollow air chambers are isolated from each other.

A substance transfer channel, a magnetic lock disposed around the substance transfer channel, and a soft switch valve matched with the magnetic lock are disposed on a side wall of the carrier chamber. The soft switch valve is a magnetized magnetic soft switch valve. The substance transfer channel is provided to allow transfer or exchange of substances between the carrier chamber and the external environment when the magnetic soft switch valve is opened. The magnetic lock and the magnetized magnetic soft switch valve are matched through a magnetic attraction force to keep the magnetic soft switch valve in a closed state, so as to ensure that the carrier chamber is isolated from the external environment when the magnetically driven capsule robot is not working.

The magnetic field-driven control module is configured to apply a magnetic torque to the magnetized magnetic soft switch valve under an action of an external magnetic field excitation source to open or close the magnetic soft switch valve.

Preferably, the substance transfer channel is one or a plurality of openings located on a side wall of the capsule shell and protruding toward the inside or outside of a capsule. A magnetic lock recess is formed between a periphery of the opening and the side wall of the capsule shell, and the magnetic lock recess is provided to fix the magnetic lock. When the magnetic soft switch valve is in a closed state, the magnetic soft switch valve covers surfaces of the magnetic lock and the substance transfer channel and is configured to close the substance transfer channel to isolate the carrier chamber from the external environment. The magnetic soft switch valve is able to deform when being controlled by the magnetic field-driven control module to cause the substance transfer channel to reach an open state and to allow transfer or exchange of substances between the carrier chamber and the external environment.

Preferably, a magnetization direction of the magnetized magnetic soft switch valve is unidirectional magnetization or symmetrical magnetization with both ends in opposite directions. During use, a magnetic torque is applied to the magnetized magnetic soft switch valve under the action of the external magnetic field excitation source, so that the magnetized magnetic soft switch valve is deformed under an action of a magnetic torque, and that the magnetic soft switch valve is opened.

Preferably, the external magnetic field excitation source is a permanent magnet or an electromagnetic coil (such as Helmholtz coil).

Preferably, the carrier chamber includes two or more independent and mutually isolated sub-carrier chambers. A side wall of each of the sub-carrier chambers is provided with the substance transfer channel, the magnetic lock disposed around the substance transfer channel, and the magnetic soft switch valve matched with the magnetic lock, so that multi-channel transfer or exchange of substances is achieved.

According to another aspect of the disclosure, the disclosure further provides a fabrication method of the magnetically driven capsule robot, and the method includes the following steps.
(1) The capsule shell is divided into two parts including a base and a top cover, and the base and the top cover of the capsule shell are fabricated through light-curing 3D printing technology.
(2) The magnetic lock is fixedly disposed on the periphery of the substance transfer channel disposed on the side wall of the carrier chamber of the capsule shell, the magnetized magnetic soft switch valve is matched with the magnetic lock, and then the base and the top cover of the magnetically driven capsule robot are packaged.

To sum up, the above technical solutions provided by the disclosure have the following beneficial effects compared with the related art.
(1) The disclosure provides a magnetically driven capsule robot with controllable drug administration and sampling functions, and the magnetically driven capsule robot includes a robot body and a magnetic field-driven control module. Herein, the robot body includes a capsule shell, two ends of the capsule shell are provided with hollow air chambers, and a carrier chamber is disposed between the hollow air chambers disposed on the two ends. A substance transfer channel, a magnetic lock disposed around the substance transfer channel, and a magnetic soft switch valve matched with the magnetic lock through a magnetic attraction force are disposed on a side wall of the carrier chamber. The internal system structure of the capsule and the method of regulating the same are both relatively simple. In the system, the controllable drug administration and sampling functions of the magnetically driven capsule robot are cleverly achieved through the bidirectional magnetic control of the magnetic attraction force and the magnetic torque. There is no complex mechanical structure inside, and the magnetic control method used is safe. The magnetically driven capsule robot provided by the disclosure is used for administration and sampling and exhibits the advantages of painlessness, non-invasion, no anesthesia, and no risk of cross-infection are provided.
(2) In the disclosure, quantitative, site-specific, and precise administration by the magnetically driven capsule robot is easy to be achieved. In a preferred embodiment of the disclosure, an external excitation magnetic field is generated by an electromagnetic coil. On the one hand, the opening angle of the magnetic soft switch valve can be controlled by adjusting the amplitude of the current in the coil, so that the size of the drug application window can be controlled. On the other hand, the frequency of the up and down flapping of the magnetic soft switch valve can be controlled by regulating the frequency of the current in the coil, so that the exchange rate of the drug with the outside can be accelerated or slowed down. Therefore, in the magnetically driven capsule robot provided by the disclosure, by adjusting the current amplitude and frequency in the excitation coil, the release of the drug can be accelerated and the release of the drug can also be slowed down. Therefore, precise release of drugs is achieved with high controllability.
(3) The method of controlling the capsule robot provided by the disclosure adopts a magnetic control method and exhibits the feature of remote wireless driving. Due to the advantages of strong magnetic field penetration and unrestricted area of action, the magnetically driven capsule robot can achieve precise quantitative and timed drug application and active sampling functions. Therefore, the drug application efficiency and administration stability are greatly improved. Besides, the double-sided administration channel design provides the possibility of window opening in any magnetic field direction and on-demand mixed administration of multiple drugs.
(4) Multifunctional reuse can be achieved. In the magnetically driven capsule robot of the disclosure, the closing and opening processes of the administration channel are independent of each other. When the drug in the administration chamber is released or the site-specific sampling function is completed, the magnetically driven capsule robot can be re-sterilized and recycled again, so the concept of resource saving is better implemented.
(5) In the magnetically driven capsule robot of the disclosure, hollow air chambers are provided at both ends of the capsule shell. The arrangement of the hollow air chambers can reduce the density of the entire magnetically driven capsule robot. Further, the overall density of the magnetically driven capsule robot can also be regulated by regulating the size of the hollow air chambers. By adjusting the dosage of the solution in the carrier chamber, the capsule robot may float and sink, it is thus ensured that the capsule robot has a strong ability to carry drugs.
(6) In the magnetically driven capsule robot of the disclosure, the magnetic lock and the magnetic soft switch valve constitute the switch of the magnetically driven capsule robot, and both of them are made of magnetic soft materials. The drug release valve or the tissue fluid suction valve of the magnetically driven capsule is closed through the magnetic attraction force between the magnetic lock and the magnetic soft switch valve. Further, combined with the regulation of the external magnetic field, the opening and closing of the magnetic soft switch valve and different deformation degrees Corresponding to different opening sizes can be achieved. Therefore, the controllable drug release, multimodal drug release, and active sampling functions of the magnetically driven capsule robot can be achieved.

### BRIEF DESCRIPTION OF THE DRAWINGS

FIG. 1 is a schematic view of a three-dimensional structure of a robot body of a magnetically driven capsule robot according to an embodiment of the disclosure, where (a) of FIG. 1 illustrates a housing of the robot body, (b) of FIG. 1 illustrates a top cover of the robot body, and 1(c) of FIG. 1 illustrates a base of the robot body.

FIG. 2 is a schematic view of working principles of the magnetically driven capsule robot when it is not performing administration and is releasing a drug according to the disclosure, where (a) of FIG. 2 is a schematic view illustrating a three-dimensional structure of a magnetically driven capsule when it is not performing administration, (b) of FIG. 2 is a schematic view illustrating the three-dimensional structure of the magnetically driven capsule when it is releasing a drug, (c) of FIG. 2 is an image of an experimental result when the magnetically driven capsule is not performing administration, and (d) of FIG. 2 is an image of an experimental result when the magnetically driven capsule is releasing a drug.

FIG. 3 is a schematic diagram of a circuit structure of magnetization and magnetizing a magnetic soft switch valve of the magnetically driven capsule robot according to an embodiment of the disclosure.

FIG. 4 is a schematic diagram of bidirectional control of the magnetic soft switch valve according to an embodiment of the disclosure, where (a) of FIG. 4 is a view illustrating a three-dimensional structure of the bidirectional magnetic soft switch valve, (b) of FIG. 4 is a working principle diagram under a forward magnetic field, and (c) of FIG. 4 is a working principle diagram under a reverse magnetic field.

FIG. 5 is schematic diagram of administration processes of the magnetically driven capsule robot under different external excitations according to an embodiment of the disclosure, where (a) of FIG. 5 shows no external magnetic field, (b) of FIG. 5 shows the presence of an external magnetic field, (c) of FIG. 5 shows the external magnetic field being turned off, and (d) of FIG. 5 shows the external magnetic field being turned on again.

### DESCRIPTION OF THE EMBODIMENTS

In order to make the objectives, technical solutions, and advantages of the disclosure clearer and more comprehensible, the disclosure is further described in detail with reference to the drawings and embodiments. It should be understood that the specific embodiments described herein serve to explain the invention merely and are not used to limit the invention.

The disclosure provides a magnetically driven capsule robot including a robot body and a magnetic field-driven control module. Herein, the robot body includes a capsule shell, and two ends of the capsule shell are provided with hollow air chambers. A carrier chamber is disposed between the hollow air chambers disposed on the two ends, and the carrier chamber and the hollow air chambers are isolated from each other. A substance transfer channel, a magnetic lock disposed around the substance transfer channel, and a magnetic soft switch valve matched with the magnetic lock are disposed on a side wall of the carrier chamber. The magnetic soft switch valve is a magnetized magnetic soft switch valve. The substance transfer channel is provided to allow transfer or exchange of substances between the carrier chamber and the external environment when the magnetic soft switch valve is opened. The magnetic lock and the magnetized magnetic soft switch valve are matched through a magnetic attraction force to keep the magnetic soft switch valve in a closed state, so as to ensure that the carrier chamber is isolated from the external environment when the magnetically driven capsule robot is not working.

The magnetic field-driven control module is configured to apply a magnetic torque to the magnetized magnetic soft switch valve under an action of an external magnetic field excitation source to open or close the magnetic soft switch valve. When working, when the magnetic soft switch valve is opened, transfer or exchange of substances may be carried out between the carrier chamber and the external environment through the substance transfer channel.

In some embodiments, the capsule shell is fabricated using light-curing 3D printing technology, and various printing methods may be used. For instance, the capsule shell may be divided into two parts: a base and a top cover, which are printed separately and then are obtained through assembly. The capsule shell is made of medical rubber, medical plastic, and other materials that exhibit good biological safety and are not affected by gastrointestinal environments such as gastric acid. For instance, MED610, transparent non-fluorescent resin materials, etc. may be used.

In the magnetically driven capsule robot provided by the disclosure, the robot body includes the capsule shell, and the two ends of the capsule shell are provided with the hollow air chambers. The carrier chamber is disposed between the hollow air chambers disposed on the two ends, and both the carrier chamber and the hollow air chambers are isolated. The carrier chamber is located inside the capsule shell. When the magnetic soft switch valve is in a closed state, the carrier chamber is a closed space, and this space is used either for the storage of drug solutions or for storing sampling tissue. The substance transfer channel disposed the magnetically driven capsule robot provided by the disclosure acts as a drug or tissue fluid exchange window between the internal carrier chamber and the external environment. In this way, when the magnetic soft switch valve is opened, a drug in the carrier chamber may reach a lesion area through the substance transfer channel, or the tissue fluid in a target area may enter the carrier chamber to achieve the function of sampling.

In some embodiments, the substance transfer channel is one or a plurality of openings located on a side wall of the capsule shell and protruding toward the inside or outside of a capsule. A magnetic lock recess is formed between a periphery of the opening and the side wall of the capsule shell, and the magnetic lock recess is provided to fix the magnetic lock. When the magnetic soft switch valve is in a closed state, the magnetic soft switch valve covers surfaces of the magnetic lock and the substance transfer channel and is configured to close the substance transfer channel to isolate the carrier chamber from the external environment. The magnetic soft switch valve is able to deform when being controlled by the magnetic field-driven control module to cause the substance transfer channel to reach an open state and to allow transfer or exchange of substances between the carrier chamber and the external environment.

In some embodiments, a magnetization direction of the magnetized magnetic soft switch valve is unidirectional magnetization or symmetrical magnetization with both ends in opposite directions, so as to facilitate the control of opening and closing of the magnetic soft switch valve. During use, a magnetic torque is applied to the magnetized magnetic soft switch valve under the action of the external magnetic field excitation source, so that the magnetized magnetic soft switch valve is deformed under an action of a magnetic torque, and that the magnetic soft switch valve is opened.

In some embodiments, when the magnetization direction is unidirectional magnetization in one direction, the deformation is a side opening with one end tilted. When the magnetization direction is symmetrical magnetization with opposite directions at both ends, the deformation is curling from both ends to the middle.

In some embodiments, a pulse magnetization module is used to magnetize the magnetic soft switch valve, and the pulse magnetization module may include a pulse power supply, a magnetizing coil, a discharge capacitor, a discharge switch, a freewheeling circuit, and a magnetization mold. Herein, the pulse power supply is configured to provide oscillation attenuation or a sinusoidal half-wave pulse current for the magnetizing coil. The magnetizing coil is configured to magnetize the magnetic soft switch valve. The discharge capacitor is configured to store electrical energy. The discharge switch is configured to trigger and turn on a discharge circuit, so that a pulse current provided by the discharge capacitor is able to flow into the magnetizing coil. The freewheeling circuit includes a freewheeling diode and a freewheeling resistor for adjusting a current waveform. The magnetization mold is configured to fix the magnetic soft switch valve. When the pulse magnetization module is working, the magnetization mold provided with the magnetic soft switch valve is disposed inside the magnetizing coil. When oscillation attenuation or a sinusoidal half-wave pulse current is introduced into the magnetizing coil, a strong pulse magnetic field is generated in an internal space of the magnetizing coil, so that the magnetic soft switch valve arranged inside the magnetization mold is subjected to non-oscillating magnetizing or oscillating de-magnetizing. Herein, when the freewheeling resistance is small (can be as low as 0), a discharge current has a non-oscillating waveform, and after flowing into the coil, the discharge current can generate a non-oscillating magnetic field for magnetizing the magnetic soft switch valve. The magnetic lock and the magnetic soft switch valve constitute a switch of the magnetically driven capsule robot of the disclosure, and both of them are made of magnetic materials.

The magnetic soft switch valve is made of a permanent magnet material at the micron scale and below (e.g., NdFeB, CᵣO₂ and other magnetic particles) and a soft material (e.g., silica gel, a TPE material, hydrogel, and other materials with elastic modulus below GPa), and is a magnetic soft composite material. The magnetic lock is made of a permanent magnet material at the micron scale and below and a non-magnetic material (e.g., silica gel, a TPE material, hydrogel, etc.). Since the magnetic lock does not require to be deformed, the selection range of both the permanent magnet material and the non-magnetic material is wider. In addition to NdFeB magnetic particles, the permanent magnet material may also be ferromagnetic particles. The non-magnetic material may be a soft material or a hard material. In some embodiments, a pre-prepared mixed solution of the magnetic lock is evenly added to a magnetic lock mold, placed in a constant temperature oven at 70° C. for solidification, and demoulded after it is completely solidified, and the fabricated magnetic lock is thus obtained. The fabrication of the magnetic soft switch valve is the same.

In the magnetically driven capsule robot of the disclosure, the magnetic lock is fixedly disposed in the magnetic lock recess on a periphery of the substance transfer channel. The surface of the magnetic lock is flush with the surface of the substance transfer channel, and the magnetic soft switch valve covers the surfaces of the magnetic lock and the substance transfer channel. The two are sealed and bonded together through magnetic attraction, thus sealing the substance transfer channel and isolating the carrier chamber from the external environment. There are two magnetic attraction modes between the magnetic lock and the magnetic soft switch valve. The magnetic lock can be magnetized or not. For instance, when the magnetic lock is made of a permanent magnet material, the magnetic lock does not need to be magnetized, so that a magnetic attraction force similar to an iron magnet may be formed between the magnetic lock and the magnetic soft switch valve. In a preferred embodiment, in order to further enhance the attraction force of the magnetic lock and the magnetic soft switch valve, the magnetic lock containing a magnetic material can be magnetized, so that an attraction effect similar to that of a permanent magnet is produced between the magnetic lock and the magnetized magnetic soft switch valve.

In some embodiments, the magnetizing coil is wound by flat copper wire, with a reinforcing material provided on its periphery, and the central area of its coil frame is hollow. In this way, when the pulse current flows through the magnetizing coil, a relatively uniform axial pulse magnetic field is generated in the central area to magnetize the magnetic soft switch valve.

In some embodiments, when symmetrically-reverse magnetization is performed on both ends of the magnetic soft switch valve through a mold method, the following steps are specifically included. The processed magnetic soft switch valve is folded symmetrically first, and then the folded magnetic soft switch valve is placed into a prefabricated magnetization mold groove. Finally, the entire magnetizing mold carrying the magnetic soft switch valve is placed in the magnetizing coil for overall axial magnetizing.

In some embodiments, the external magnetic field excitation source is a permanent magnet or an electromagnetic coil (such as Helmholtz coil).

In some embodiments, the external magnetic field excitation source applies a magnetic torque of 50 Hz or below and 100 mT or below to the magnetized magnetic soft switch valve, so that the magnetized magnetic soft switch valve is deformed under an action of a magnetic torque, and that the magnetic soft switch valve is opened.

In some embodiments, the carrier chamber includes two or more independent sub-carrier chambers. A side wall of each of the sub-carrier chambers is provided with the substance transfer channel, the magnetic lock disposed around the substance transfer channel, and the magnetic soft switch valve matched with the magnetic lock, so that multi-channel transfer or exchange of substances is achieved.

In a preferred embodiment, the carrier chamber includes sub-carrier chambers disposed symmetrically up and down and thus is symmetrically provided with the substance transfer channels, the magnetic locks disposed around the substance transfer channels, and the magnetic soft switch valves matched with the magnetic locks. In this way, bilateral transfer or exchange of substances may be achieved, making it possible to mix multiple drugs.

The disclosure further provides a fabrication method of the magnetically driven capsule robot, and the method includes the following steps.
(1) The robot body of the magnetically driven capsule robot provided by the disclosure is divided into two parts including the base and the top cover, and the base and the top cover of the capsule shell are fabricated through light-curing 3D printing technology.
(2) The magnetic lock is fixedly disposed on the periphery of the substance transfer channel disposed on the side wall of the carrier chamber of the capsule shell, the magnetized magnetic soft switch valve is matched with the magnetic lock, and then the base and the top cover of the magnetically driven capsule robot are packaged to obtain the magnetically driven capsule robot.

In some embodiments, the magnetic lock is embedded into the magnetic lock recess formed by the periphery of the substance transfer channel and the side wall of the capsule shell to be fixed. To be specific, in the magnetically driven capsule robot provided by the disclosure, the substance transfer channel disposed on the capsule shell may be one or a plurality of openings located on the side wall of the carrier chamber of the capsule shell and protruding toward the inside of the capsule or may be one or a plurality of openings located on the side wall of the carrier chamber protruding toward the outside of the capsule. When the opening is configured to protrude inwards, the periphery of the opening and an inner wall of the capsule shell form the magnetic lock recess, and the magnetic lock is embedded into the magnetic lock recess formed by the periphery of the substance transfer channel and the inner wall of the capsule shell to be fixed. When the opening is configured to protrude outwards, the periphery of the opening and an outer wall of the capsule shell form the magnetic lock recess, and the magnetic lock is embedded into the magnetic lock recess formed by the periphery of the substance transfer channel and the outer wall of the capsule shell to be fixed.

In some embodiments, in step (2), the discharge capacitor in the pulse magnetization module is charged first, and then the discharge switch is triggered to discharge the magnetizing coil. A uniform axial magnetic field is generated in the central area of the coil to axially fully magnetize the folded magnetic soft switch valve placed in the magnetization mold, and a magnetic soft switch valve with reversely symmetrical magnetization distribution at both ends is thus obtained.

In some embodiments, in order to improve the controllability of the magnetic soft switch valve, the magnetizing voltage of the pulse power supply may be increased to ensure that the magnetized magnetic soft switch valve has higher residual magnetization characteristics, so that the breaking capacity of the magnetic soft switch valve is improved.

A method of using the magnetically driven capsule robot provided by the disclosure to perform controllable drug administration specifically includes the following steps. When the magnetically driven capsule robot containing a drug reaches the lesion, the drug is not released due to the magnetic attraction force of the magnetic lock to the magnetic soft switch valve. Herein, the external magnetic field excitation source is used to apply a magnetic field outside the human body, so that both ends of the magnetic soft switch valve are acted upon by the magnetic torque. The magnetic soft switch valve opens to release the drug solution to the lesion for administration. When the drug release is completed or the drug reaches the released amount, the magnetic field is reversed and drug administration is stopped. Further, the magnetic soft switch valve may also be repeatedly closed and opened by repeatedly changing the magnetic field direction of the external magnetic field excitation source in the magnetic field-driven control module, and the full release of the drug is thereby accelerated.

A method of sampling using the magnetically driven capsule robot provided by the disclosure includes the following steps. When the magnetically driven capsule robot reaches a sampling target area, the external magnetic field excitation source is used to apply a magnetic field outside the human body, causing both ends of the magnetic soft switch valve to bend and deform inward under the action of the magnetic torque. The substance transfer channel is opened, allowing tissue fluid in a designated area to enter the carrier chamber. When sufficient tissue fluid is extracted from the carrier chamber, the magnetic field of the magnetic field-driven control module is reversed, so that the magnetic soft switch valve is closed, and the sampling function of the designated area is thereby completed.

In the disclosure, when the magnetically driven capsule robot is used for controllable drug administration, the substance transfer channel is an administration channel, and the carrier chamber is a drug-carrying chamber. The magnetic soft switch valve provided therein is used to release the drug inside the magnetically driven capsule robot and is disposed between the administration channel and the drug-carrying chamber. When the magnetic soft switch valve is in a closed state, that is, the magnetic soft switch valve is affected by a magnetic attraction force of the magnetic lock, thereby ensuring that the drug inside the magnetically driven capsule does not exchange with the outside. When the external magnetic field of the magnetic field-driven control module acts, the magnetic torque experienced by the magnetic soft switch valve is greater than the magnetic attraction force generated by the magnetic lock. At this time, the magnetic soft switch valve changes from the closed state to an open state, so the drug in the drug-carrying chamber may reach the lesion area in the human body through the administration channel. Further, in order to accelerate the release of drugs within human tissues, the magnetic soft switch valve may be closed and opened by repeatedly changing the magnetic field direction of the magnetic field-driven control module. The flapping effect of the valve is used to accelerate the exchange of drug solution and internal tissue fluid of the human body, so that administration efficiency is improved to a significant extent.

On the other hand, if the magnetically driven capsule robot is used for active sampling, the function of the magnetic soft switch valve is to open the substance transfer channel when the magnetically driven capsule reaches the lesion area, tissue fluid in the designated area is thereby extracted. When the magnetic field-driven control module is not working, the magnetic soft switch valve is affected by the magnetic attraction force of the magnetic lock, thereby ensuring that the interior of the carrier chamber is isolated from the external environment. When the magnetic field-driven control module applies an external magnetic field, the magnetic soft switch valve bends and deforms inward due to the effect of magnetic torque, and the substance transfer channel opens, thereby allowing the tissue fluid in the designated area to enter the carrier chamber. When sufficient tissue fluid is extracted from the carrier chamber, the external magnetic field of the magnetic field-driven control module is reversed, so that the magnetic soft switch valve is closed, and the sampling function of the designated area is thereby completed.

In the disclosure, the methods used by the magnetically driven capsule robot to achieve site-specific administration and sampling may be achieved by using imaging equipment assistance, magnetic positioning, or gastrointestinal motility timing monitoring, and other methods conventionally used in the art to achieve site-specific administration.

In the disclosure, the internal system structure and the method of regulating the magnetically driven capsule are simple. The on-demand and targeted administration and sampling functions of the magnetically driven capsule are cleverly achieved through the bidirectional magnetic control of the magnetic attraction force and the magnetic torque. Further, the administration progress may be accelerated and slowed down by controlling the amplitude and frequency of the external excitation field current. Besides, the double-sided administration channel design provides the possibility of window opening in any magnetic field direction and on-demand mixed administration of multiple drugs. Combined with the advantages of strong magnetic control penetrating ability and unrestricted area of action, the magnetically driven capsule robot provided by the disclosure provides a new technical path for the multi-functionalization of the capsule robots.

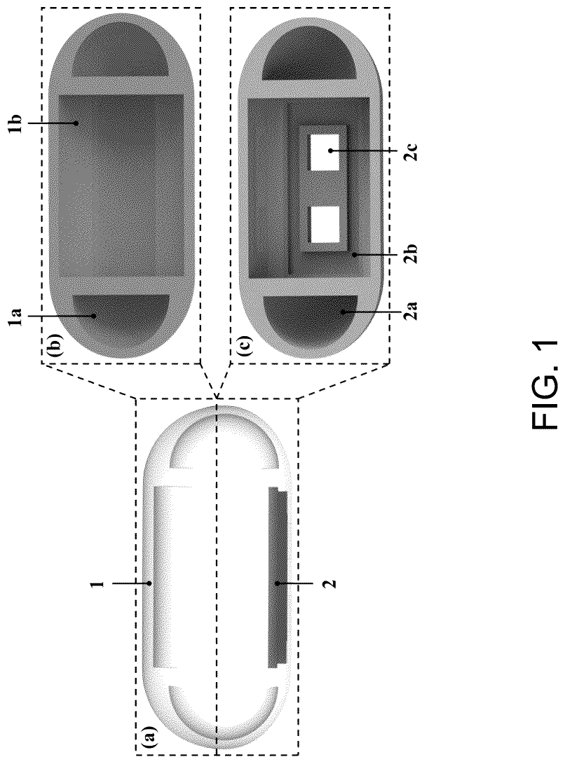

In some embodiments, as shown in FIG. 1, the magnetically driven capsule robot used for controllable drug administration includes the robot body, and the robot body includes the capsule shell. During fabrication, SolidWorks software is used first to build a three-dimensional model of the capsule shell, the three-dimensional model is exported as an STL file, and then the exported file is put into slicing software for processing and put into a 3D printer for printing. Considering the safety of the capsule robot, the capsule shell is printed using MED610, a material with good biocompatibility. In order to facilitate the assembly of the capsule robot, the capsule shell is divided into upper and lower portions, consisting of a top cover 1 and a base 2, as shown in (a) of FIG. 1. After the magnetic soft switch valve and the magnetic lock are assembled to the base 2, the upper and lower portions of the capsule shell are sealed. Herein, the top cover 1 has a semi-ellipsoid structure and is formed by two hemispherical hollow air chambers la and a semi-cylindrical administration chamber 1b, as shown in (b) of FIG. 1. As shown in (c) of FIG. 1, the base 2 is formed by two hemispherical hollow air chambers 2a, a magnetic lock groove 2b, and administration channels 2c. There are two administration channels 2c (i.e., substance transfer channels), which are openings provided on the inner wall of the capsule shell and protruding toward the inside of the capsule. A recess is formed on the periphery of the openings and the inner wall of the capsule shell, that is, the magnetic lock groove 2b.

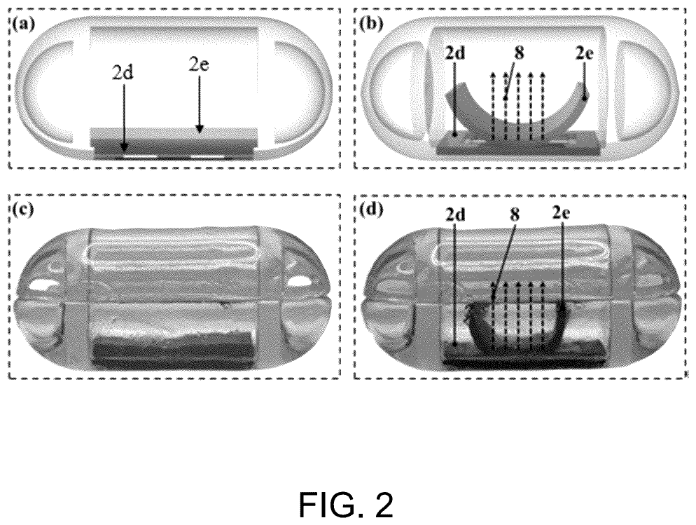

As shown in (a) of FIG. 2, a magnetic lock 2d is embedded in the magnetic lock groove 2b to be fixed. A magnetized magnetic soft switch valve 2e covers the surfaces of the magnetic lock 2d and the substance transfer channel (i.e., administration channel 2c) and is configured to close the administration channel 2c to isolate the administration chamber (i.e., carrier chamber) from the external environment. The magnetic soft switch valve 2e may be curled inward from both ends to reach the open state under the control of the magnetic field-driven control module, as shown in (b) of FIG. 2, and is configured to open the substance transfer channel, that is, the administration channel 2c, so that the drug in the administration chamber is released to the external environment for drug transfer. The top cover 1 and the base 2 together form the capsule shell with a capsule shape, and the capsule shell is made of the transparent resin material MED610. The arrangement of the hollow air chambers in the capsule structure is to reduce the density of the entire magnetically driven capsule robot. Further, by adjusting the dosage of the solution in the carrier chamber, the capsule robot may float and sink, it is thus ensured that the capsule robot has a strong ability to carry drugs.

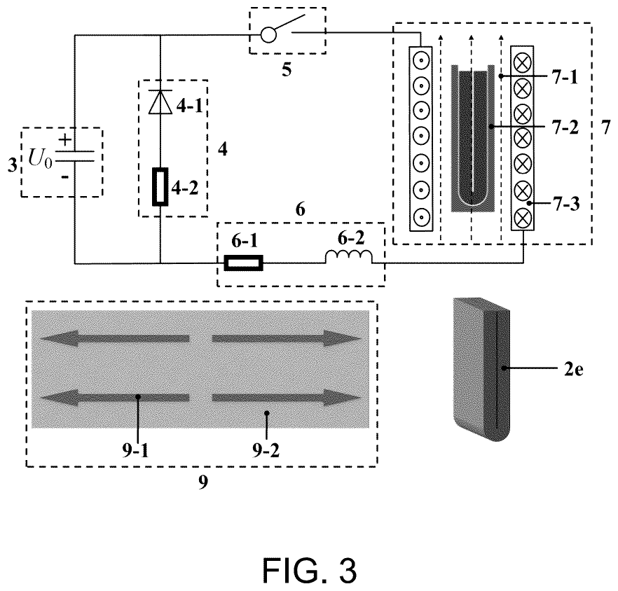

In some embodiments, the pulse magnetization module as shown in FIG. 3, that is, a magnetizing circuit structure, is used to achieve symmetrical magnetization of the magnetic soft switch valve. A discharge capacitor 3, a discharge switch 5, a magnetizing coil 7-3, line impedance 6 (including a line resistor 6-1 and an inductor 6-2), a freewheeling circuit 4 (including a diode 4-1 and a freewheeling resistor 4-2), and a magnetization mold 7-2 are specifically included. As shown in FIG. 3, before discharge, the magnetic soft switch valve 2e is symmetrically folded to obtain the magnetic soft switch valve 2e in a folded state and is then placed inside the magnetization mold 7-2. Next, the magnetization mold 7-2 and the magnetic soft switch valve 2e are placed as a whole in the magnetizing coil 7-3 for magnetizing. Further, the capacitor 3 is charged, and then the discharge switch 5 is closed to discharge the magnetizing coil 7-3. Based on the above, the central area of the magnetizing coil 7-3 generates a uniform pulse magnetic field 7-1, which allows the particles in the magnetic soft switch valve to form a radial magnetization distribution characteristic as shown in 9 of FIG. 3 (the magnetization directions are shown in 9-1 and 9-2).

The structural schematic diagram of the magnetically driven capsule robot fabricated in the experiment before releasing the drug and after releasing the drug is respectively shown in (c) and (d) of FIG. 2. First, the robot body of the magnetically driven capsule robot reaches the lesion through the peristaltic action of the human intestines and stomach. Due to the electromagnetic attraction force of the magnetic lock 2d to the magnetic soft switch valve 2e, the drug is in an unreleased state, and the three-dimensional structural diagram and the experimental result are respectively shown in (a) and (c) of FIG. 2. At this time, a magnetic field 8 is applied outside the human body through the electromagnetic coil (such as Helmholtz coil), so that both ends of the magnetic soft switch valve 2e are acted upon by the magnetic torque. The magnetic soft switch valve opens to release the drug solution to the lesion for administration, and the three-dimensional structure diagram and experimental result are respectively shown in (b) and (d) of FIG. 2. When the drug release is completed or the drug reaches the released amount, the magnetic field is reversed and drug administration is stopped. Further, the magnetic soft switch valve may also be repeatedly closed and opened by repeatedly changing the magnetic field direction of the control module, so that the full release of the drug is accelerated.

In some embodiments, the external magnetic field 8 used in the magnetic field-driven control module to open and close the magnetic soft switch valve is generated by the electromagnetic coil. Preferably, when the magnetically driven capsule robot reaches the designated location and needs to release the drug, a sinusoidal pulse current is introduced into the electromagnetic coil, an oscillating magnetic field with alternating directions is thereby generated to repeatedly open and close the magnetic soft switch valve. At the same time, the repeated oscillation of the magnetic soft switch valve causes the surrounding liquid to accelerate the flow, and the drug release efficiency is thereby further accelerated.

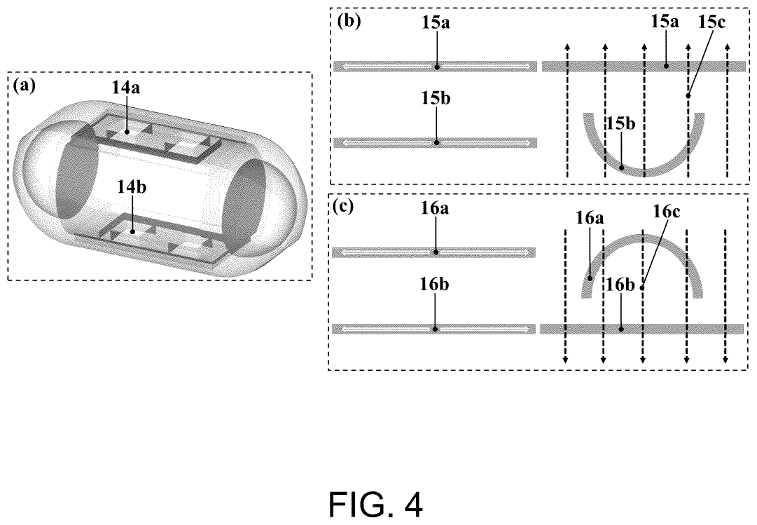

In some embodiments, the magnetically driven capsule robot is further designed into a double-sided administration channel. That is, in addition to providing the administration channel, the magnetic lock, and the magnetic soft switch valve on the base of the capsule shell, a corresponding set is also provided on the upper top cover. In FIG. 4, (a) is a schematic structural diagram illustrating the dual-channel robot body, (b) is a working principle diagram in an upward magnetic field direction, and (c) is a working principle diagram in a downward magnetic field direction, where 14a is an upper-side administration channel, and 14b is a lower-side administration channel. Such a design can further improve the flexibility and controllability of the magnetically driven capsule robot. To be specific, when an upper-side magnetic soft switch valve 15a and a lower-side magnetic soft switch valve 15b are excited by an external magnetic field 15c in an upward direction, the upper-side magnetic soft switch valve 15a is in a closed state due to the restraint effect of the capsule shell, while the lower-side magnetic soft switch valve 15b causes the lower-side administration channel to open for drug release due to the action of the magnetic torque. On the contrary, when an upper-side magnetic soft switch valve 16a and a lower-side magnetic soft switch valve 16b are excited by an external magnetic field 16c in a downward direction, the upper-side magnetic soft switch valve 16a causes the upper-side administration channel to open for drug release due to the action of the magnetic torque, while the lower-side magnetic soft switch valve 16b is in a closed state due to the restraint effect of the capsule shell. Based on the above analysis, it can be seen that this design of a double-sided administration channel ensures that the magnetically driven capsule robot always has one side of the administration channel open regardless of whether it is under the action of a forward magnetic field or a reverse magnetic field. This structural design can further improve the drug application efficiency of the administration capsule. Further, when a baffle is provided between the top cover 1 and the base 2 of the capsule shell to isolate the upper administration chamber from the lower administration chamber, different drugs may be placed in the upper and lower administration chambers, making it possible to mix and administer different drugs.

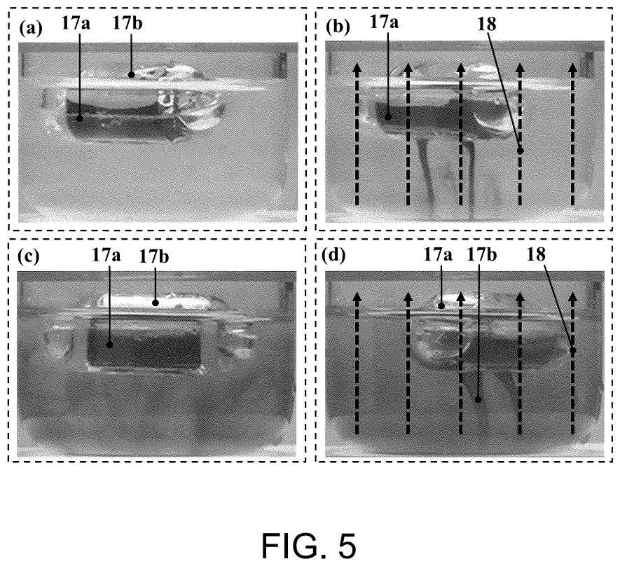

In some other embodiments, in order to verify the practicability of the magnetically driven capsule robot, its simulated administration experiment diagram is shown in FIG. 5. (a) of FIG. 5 shows the magnetically driven capsule robot carrying a drug being placed into clean water. It can be seen that when no external magnetic field is applied, a drug 17a in a magnetically driven capsule 17b is not released into the clean water. When an external magnetic field 18 is applied, the drug 17a is released through the administration channel, as shown in (b) of FIG. 5. Further, when the external magnetic field 18 is turned off, it can be seen from (c) of FIG. 5 that the administration channel of the magnetically driven capsule 17b is closed, and no obvious penetration of the drug 17a occurs. When the external magnetic field 18 is further turned on, the drug 17a in the magnetically driven capsule 17b is released into the external clean water environment again. as shown in (d) of FIG. 5. This drug administration simulation experiment shows that the magnetically driven capsule robot provided by the disclosure is expected to achieve accurate quantitative and timed administration and exhibits high controllability.

In some other embodiments, the abovementioned magnetically driven capsule robot is used for active sampling. When the magnetically driven capsule robot reaches the sampling target area, the external magnetic field excitation source is used to apply a magnetic field outside the human body, causing both ends of the magnetic soft switch valve to bend and deform inward under the action of the magnetic torque. The substance transfer channel is opened, allowing the tissue fluid in the designated area to enter the carrier chamber. When sufficient tissue fluid is extracted from the carrier chamber, the magnetic field of the magnetic field-driven control module is reversed, so that the magnetic soft switch valve is closed, and the sampling function of the designated area is thereby completed.

The above results fully prove that in the disclosure, through the bidirectional magnetic control of the magnetic attraction force and the magnetic torque, the on-demand and site-specific administration and sampling functions of the magnetically driven capsule can be achieved. The structure of the administration device is simple and lightweight. The magnetically driven capsule may be opened and closed remotely and repeatedly through an alternating magnetic field. Advantages such as wireless driving and strong controllability are provided, so the disclosure exhibits potential medical values.

A person having ordinary skill in the art should be able to easily understand that the above description is only preferred embodiments of the disclosure and is not intended to limit the disclosure. Any modifications, equivalent replacements, and modifications made without departing from the spirit and principles of the disclosure should fall within the protection scope of the disclosure.

What is claimed is:

1. A magnetically driven capsule robot, comprising a robot body and a magnetic field-driven control module, wherein
the robot body comprises a capsule shell, two ends of the capsule shell are provided with hollow air chambers, a carrier chamber is disposed between the hollow air chambers, and the carrier chamber and the hollow air chambers are isolated from each other,
a substance transfer channel, a magnetic lock disposed around the substance transfer channel, and a magnetic soft switch valve matched with the magnetic lock are disposed on a side wall of the carrier chamber, the magnetic soft switch valve is a magnetized magnetic soft switch valve, the substance transfer channel is provided to allow transfer or exchange of substances between the carrier chamber and an external environment when the magnetic soft switch valve is opened, and the magnetic lock and the magnetized magnetic soft switch valve are matched through a magnetic attraction force to keep the magnetic soft switch valve in a closed state, so as to ensure that the carrier chamber is isolated from the external environment when the magnetically driven capsule robot is not working,
the magnetic field-driven control module is provided to apply a magnetic torque to the magnetized magnetic soft switch valve under an action of an external magnetic field excitation source to open or close the magnetic soft switch valve.

2. The magnetically driven capsule robot according to claim 1, wherein the substance transfer channel is one or a plurality of openings located on a side wall of the capsule shell and protruding toward an inside or an outside of a capsule, a magnetic lock recess is formed between a periphery of the opening and the side wall of the capsule shell, and the magnetic lock recess is provided to fix the magnetic lock,
when the magnetic soft switch valve is in a closed state, the magnetic soft switch valve covers surfaces of the magnetic lock and the substance transfer channel and is configured to close the substance transfer channel to isolate the carrier chamber from the external environment, and the magnetic soft switch valve is able to deform when being controlled by the magnetic field-driven control module to cause the substance transfer channel to reach an open state and to allow transfer or exchange of the substances between the carrier chamber and the external environment.

3. The magnetically driven capsule robot according to claim 2, wherein a magnetization direction of the magnetized magnetic soft switch valve is unidirectional magnetization or symmetrical magnetization with both ends in opposite directions,
during use, a magnetic torque is applied to the magnetized magnetic soft switch valve under the action of the external magnetic field excitation source, so that the magnetized magnetic soft switch valve is deformed under an action of the magnetic torque, and that the magnetic soft switch valve is opened.

4. The magnetically driven capsule robot according to claim 3, wherein a pulse magnetization module is used to magnetize the magnetic soft switch valve, and the pulse magnetization module comprises a pulse power supply, a magnetizing coil, a discharge capacitor, a discharge switch, a freewheeling circuit, and a magnetization mold, wherein
the pulse power supply is configured to provide oscillation attenuation or a sinusoidal half-wave pulse current for the magnetizing coil, the magnetizing coil is configured to magnetize the magnetic soft switch valve, the discharge capacitor is configured to store electrical energy, the discharge switch is configured to trigger and turn on a discharge circuit, so that a pulse current provided by the discharge capacitor is able to flow into the magnetizing coil, the freewheeling circuit comprises a freewheeling diode and a freewheeling resistor for adjusting a current waveform, the magnetization mold is configured to fix the magnetic soft switch valve,
when the pulse magnetization module is working, the magnetization mold internally provided with the magnetic soft switch valve is arranged inside the magnetizing coil, and when the oscillation attenuation or the sinusoidal half-wave pulse current is introduced into the magnetizing coil, a strong pulse magnetic field is generated in an internal space of the magnetizing coil, so that the magnetic soft switch valve arranged inside the magnetization mold is subjected to oscillating de-magnetizing or non-oscillating magnetizing.

5. The magnetically driven capsule robot according to claim 4, wherein when the unidirectional magnetization is performed to magnetize the magnetic soft switch valve through a mold method, the following steps are specifically comprised: placing the magnetic soft switch valve that is processed into a prefabricated magnetization mold and entirely placing a magnetizing mold carrying the magnetic soft switch valve into the magnetizing coil to perform overall axial magnetizing to obtain the magnetic soft switch valve whose magnetization direction is unidirectional magnetization,
when symmetrically-reverse magnetization is performed to magnetize the both ends of the magnetic soft switch valve through the mold method, the following steps are specifically comprised: symmetrically folding the magnetic soft switch valve that is processed first, placing the magnetic soft switch valve that is folded into the prefabricated magnetization mold, and finally, placing the magnetizing mold carrying the magnetic soft switch valve into the magnetizing coil entirely for overall axial magnetizing to obtain a symmetrically-magnetized magnetic soft switch valve with the both ends in opposite magnetization directions.

6. The magnetically driven capsule robot according to claim 1, wherein the external magnetic field excitation source is a permanent magnet or an electromagnetic coil.

7. The magnetically driven capsule robot according to claim 1, wherein the carrier chamber comprises two or more independent and mutually isolated sub-carrier chambers, and a side wall of each of the sub-carrier chambers is provided with the substance transfer channel, the magnetic lock disposed around the substance transfer channel, and the magnetic soft switch valve matched with the magnetic lock, so that multi-channel transfer or exchange of the substances is achieved.

8. A fabrication method of the magnetically driven capsule robot according to claim 1, comprising:
(1) dividing the capsule shell into two parts comprising a base and a top cover and fabricating the base and the top cover of the capsule shell through light-curing 3D printing technology; and
(2) fixedly arranging the magnetic lock on a periphery of the substance transfer channel disposed on the side wall of the carrier chamber of the capsule shell, matching the magnetized magnetic soft switch valve with the magnetic lock, and then packaging the base and the top cover of the magnetically driven capsule robot to obtain the magnetically driven capsule robot.

9. The fabrication method according to claim 8, wherein a material of the capsule shell is a medical polymer material, preferably medical rubber or medical plastic.

10. The fabrication method according to claim 8, wherein the magnetic lock is made of a magnetic material, and the magnetic soft switch valve is made of a magnetic soft material.
